(19) United States
(12) Patent Application Publication
(10) Pub. No.: US 2023/0197209 A1
(43) Pub. Date: Jun. 22, 2023

(54) GRAPH BASED MACHINE LEARNING FOR GENERATING VALID SMALL MOLECULE COMPOUNDS

(71) Applicant: Chengdu Anticancer Bioscience, LTD., Chengdu (CN)

(72) Inventors: Hongyang Yu, Sydney (AU); Hongjiang Yu, Sydney (AU)

(21) Appl. No.: 17/954,969

(22) Filed: Sep. 28, 2022

Related U.S. Application Data
(60) Provisional application No. 63/291,552, filed on Dec. 20, 2021.

(51) Int. Cl. G16C 20/70; G06N 3/08; G16C 20/50

(52) U.S. Cl. CPC G16C 20/70; G06N 3/08; G16C 20/50

(57) ABSTRACT

Disclosed herein is an automated small molecule generation process for use in in silico drug discovery. The automated process employs a trained neural network that analyzes a graph adjacency tensor which represents a small molecule compound. Over subsequent iterations, the trained neural network analyzes the graph adjacency tensor and predicts actions (e.g., adding an atom, adding a bond type, or assigning a charge) that, if taken, are likely to lead to a valid small molecule compound. Thus, the methods described herein generate small molecule compounds of increased validity in comparison to conventional methodologies.

### CROSS REFERENCE TO RELATED APPLICATION

This application claims the benefit of and priority to U.S. Provisional Patent Application No. 63/291,552 filed Dec. 20, 2021, the entire disclosure of which is hereby incorporated by reference in its entirety for all purposes.

### BACKGROUND OF THE INVENTION

Automated molecule generation is a valuable step for in-silico drug discovery processes. Algorithms for automated molecule generation have seen significant progress over recent years. However, they are often complex to implement, hard to train, and under-perform when generating long-sequence molecules. The development of a simple and powerful alternative can help accelerate drug discovery.

### SUMMARY

Conventional drug discovery is a painstaking and costly process that could take years to generate target molecules with desired properties. Over the recent years, the prevailing application of information technology in various industries has yielded massive amount of valuable data for machine learning (ML) applications. For the pharmaceutical industry, the creation of molecule database such as ChEMBL establishes the feasibility of training sophisticated algorithms to enable automation of part of or possibly the entire drug discovery process. Such an advance can shorten the time and resources required for generating potent target molecules. Deep learning as one of the focused fields of ML has become a popular backbone for designing algorithms for automated drug discovery.

Despite these great success, automated molecule generation still remains a challenging task, mainly due to the need to search through a massive and discrete space of atom types, bond types and possible connection edges. It has been reported in the work of Polishchuk et al. that the search space could be as large as 10³³. Representing molecular structure in a compact form is essential to formulating solution to this challenge. The SMILES representation of molecular structures is a promising candidate to achieve this.

SMILES compactifies molecular graphs into strings. Graph generation can therefore be reformulated as a sequential string generation process. A major advantage of this reformulation is a highly compact action space, enabling SMILES-based generation methods to generate long-sequence and unique molecules. However, strings generated intermittently are not guaranteed to be chemically valid, as they may not correspond to a meaningful substructure of a molecular graph. In this respect, direct graph generation is an attractive alternative; it is possible to validate each generation step by using chemical rules to ensure meaningful substructure generation.

Existing graph generation algorithms may be broadly classified into two categories: one-shot and sequential. One-shot algorithms aim at generating the entire molecular graph in a single forward pass of a generative model. One-shot generation methods can generate a novel molecule but typically need to analyze a huge search space, which requires significant resources (e.g., computational resources and time). GraphVAE, RVAE, GraphNVP and GRF are representative cases in this respect. GraphVAE and RVAE both attempt to learn and generate molecular graph using variational autoencoders. GraphNVP and GRF are invertible flow-based methods developed to avoid the need to train the decoder part of autoencoders, thereby reducing the number of model parameters. Regardless, the main goal is to learn a continuous latent representation that can be decoded to a valid molecular graph. Notably, this type of graph learning is not permutation invariant making it difficult to create a sensible loss function for training. For this reason, expensive approximation such as graph matching might be needed. In addition, as the size of the molecular graph increases, the number of nodes (atoms) and edges (bonds) needed to be generated in one-shot increase quadratically, which can lead to model collapse due to inadequate learning and generalization capacity. As a result, significant performance drop has all been observed in these four algorithms when switching from QM9 database with shorter length molecules to the ZINC database containing molecules with much longer length.

Sequential graph generation algorithms can relax the capacity limitation of their one-shot counterparts to some degree. A single node/edge is generated per step conditioned on already generated subgraph. Such a sequential graph generation method also enables chemical validity check per generation step leading to improved validity rate. A number of recent works including DeepGMG, GCPN, HierVAE, and GraphAF all follow this paradigm. A common feature among these works is the prevailing use of spatial graph convolutional neural (GCN) network as the backbone for graph generation, and the maximization of likelihood of training data as the training objective. The main role of graph convolutional neural network is to propagate node and edge information throughout the graph by means of consecutive graph convolution. As the number of nodes/edges increases, the number of propagation steps also need to increase to ensure complete propagation of information. This can reduce the scalability of this class of generation algorithms. Additionally, in the aforementioned works, a separate multi-layered perceptron is generally needed to produce embedding representations of nodes and edges independently, which also adds to the complexity of the generation model, making stable end-to-end training difficult to attain without careful tuning of hyper parameters.

Disclosed herein is a machine learning algorithm that is easy to train, easy to deploy, and cheap to host. These are all benefits that enable the rapid and efficient implementation of the machine algorithm (e.g., implementation as a service). Simultaneously, the machine learning algorithm achieves competitive performance while achieving the improved ease in training and deployment.

In particular embodiments, disclosed herein is a trained machine learning model to directly predict the next bond or edge to be added to a graph adjacency tensor e.g., based on a subgraph of the graph adjacency tensor. Additionally, a simple restriction mechanism is implemented to curb the growth of action space/output size of the model. For example, once a new atom is created, no modification of bonds between existing atoms is allowed, and equivalently, only bonds can be created between the new atom and other existing atoms. This restriction reduces the growth of the action space from a quadratic one to a linear one, making it easier to generate long-sequence molecules that retain their likely validity.

During training of a machine learning model, training data is generated by decomposing a molecular graph into sequences of subgraph-action pairs. The subgraph-action pairs are randomly shuffled to break autocorrelation, thereby enabling the training of the machine learning model on different variations of the decomposed molecule. This ensures that for a particular subgraph (e.g., portion of a graph adjacency tensor), the machine learning model predicts an action that, if taken, would lead to a new, valid subgraph that incorporates the action. Thus, instead of predicting the best action that would lead to the validity of an overall compound, the machine learning model predicts the best action that leads to the validity of a new subgraph representing only a portion of the overall compound.

Unlike aforementioned graph-based algorithms, the pretrained model disclosed herein operates directly on the graph adjacency tensor of a molecule and outputs a single probability distribution over all allowed atom and bond creation actions. Such an end-to-end model is simple to implement and easy to train. The main advantages the disclosed model and implementation are as follows:
Simplicity: The model input is a fixed dimension adjacency tensor of a chemical graph similar to a multiple channel input image commonly used in computer vision. This model produces a single output distribution that can be directly sampled to determine whether a new bond and atom need to be added to the subgraph;
Scalability: The model operates directly on adjacency tensor. The width of the adjacency tensor corresponds to the number of atoms in a molecule. A molecule with 100 atoms would use a tensor of size 100×100×d where d<100 is the depth of the feature channel. This is considered a relatively small-size input as compared to high resolution image inputs used in computer vision applications. Results in the Examples section below show that the disclosed model can easily handle molecules with longer sequences, and the model's performance improves with larger datasets;
Stability: The stability of the ResNet model during training has been well established. Here, only minimal hyper-parameter tuning is conducted to achieve competitive and improved results as compared to other state-of-the-art baselines.

Disclosed herein is a method for generating one or more small molecule compounds, the method comprising: obtaining a graph adjacency tensor, wherein the adjacency tensor comprises a representation of a small molecule compound; iteratively applying a trained machine learning model to generate the small molecule compound, wherein each iteration comprises: analyzing, using the machine learning model, the graph adjacency tensor to generate probabilities corresponding to available actions, wherein each probability indicates a likelihood of generating a valid substructure of a small molecule compound if a corresponding available action were taken; selecting one of the available actions based on the probabilities; updating the graph adjacency tensor with a value indicative of the selected action to generate an updated graph adjacency tensor, wherein the trained machine learning model is trained using training examples indicating actions at individual steps of the small molecule building process.

Additionally disclosed herein is a method for generating one or more small molecule compounds, the method comprising: obtaining a graph adjacency tensor, wherein the adjacency tensor comprises a representation of a small molecule compound; iteratively applying a trained machine learning model to generate the small molecule compound, wherein each iteration comprises: analyzing, using the machine learning model, a subgraph of the graph adjacency tensor to generate probabilities corresponding to available actions, the subgraph representing a substructure of the small molecule compound, wherein each probability indicates a likelihood of generating a valid substructure of a small molecule compound if a corresponding available action were taken, wherein the available actions comprise adding an atom, adding a bond, or assigning a charge to an atom; selecting one of the available actions based on the probabilities; updating the graph adjacency tensor with a value indicative of the selected action to generate an updated graph adjacency tensor, wherein the trained machine learning model is trained using training examples indicating actions at individual steps of the small molecule building process, wherein the training examples are generated by: obtaining a plurality of training small molecule compounds; for each of one or more training small molecule compounds of the plurality, generating a plurality of subgraph-action pairs for the training small molecule compound by: decomposing the training small molecule compound into a sequence of actions for generating the training small molecule compound; generating graph adjacency tensor subgraphs for actions in the sequence of actions; and pairing each action with a corresponding graph adjacency tensor subgraph; randomly shuffling subgraph-action pairs to break autocorrelations; and assigning randomly shuffled subgraph-action pairs to the training examples.

In various embodiments, the available actions comprise adding an atom, adding a bond, or assigning a charge to an atom. In various embodiments, the iterative application of the trained machine learning model terminates after the small molecule compound comprises at least a threshold number of atoms. In various embodiments, the threshold number of atoms is at least 5, at least 10, at least 20, at least 30, at least 40, or at least 50 atoms. In various embodiments, selecting one of the available actions based on the probabilities comprises selecting an available action corresponding to a highest probability. In various embodiments, the selected action comprises adding an atom, and wherein updating the graph adjacency tensor with a value indicative of the selected action comprises updating a diagonal of the graph adjacency tensor with a value indicative of the added atom. In various embodiments, the selected action comprises adding a bond, and wherein updating the graph adjacency tensor with a value indicative of the selected action comprises updating an upper portion of the graph adjacency tensor with a value indicative of the added bond. In various embodiments, the selected action comprises assigning a charge to an atom, and wherein updating the graph adjacency tensor with a value indicative of the selected action comprises updating a diagonal of the graph adjacency tensor with a value indicative of the assigned charge.

In various embodiments, the graph adjacency tensor comprises m×n×d dimensions. In various embodiments, m or n represents a pre-determined maximum number of atoms of the small molecule compound. In various embodiments, m is between 20 and 60. In various embodiments, n is between 20 and 60. In various embodiments, d is between 20 and 30. In various embodiments, analyzing, using the trained machine learning model, the graph adjacency tensor to generate probabilities corresponding to available actions further comprises: determining a subgraph of the graph adjacency tensor, the subgraph representing a substructure of the small molecule compound; and analyzing the subgraph using the trained machine learning model to predict probabilities corresponding to available actions. In various embodiments, each of the dimensions p×q×r of the subgraph are smaller than or equal to each of corresponding dimensions m×n×d of the graph adjacency tensor. In various embodiments, p is between 1 and 5. In various embodiments, q is between 1 and 5. In various embodiments, r is between 1 and 5.

In various embodiments, the training examples used to train the machine learning model are generated by: obtaining a plurality of small molecule compounds; for each of one or more small molecule compounds of the plurality, generating a plurality of subgraph-action pairs for the small molecule compound by: decomposing the small molecule compound into a sequence of actions for generating the small molecule compound; generating graph adjacency tensor subgraphs for actions in the sequence of actions; and pairing each action with a corresponding graph adjacency tensor subgraph; randomly shuffling subgraph-action pairs to break autocorrelations; and assigning randomly shuffled subgraph-action pairs to training examples. In various embodiments, the trained machine learning model is trained using at least 1 million training examples. In various embodiments, the trained machine learning model is trained using at least 10 million training examples. In various embodiments, the trained machine learning model is trained using at least 100 million training examples.

In various embodiments, obtaining a plurality of small molecule compounds comprises obtaining the plurality of small molecule compounds from a publicly available dataset. In various embodiments, the publicly available dataset is any of a QM9, ZINC250k, or ChEMBL dataset. In various embodiments, at least 90% of the generated small molecule compounds are chemically valid. In various embodiments, at least 95%, at least 96%, at least 97%, at least 98%, or at least 99% of the generated small molecule compounds are chemically valid. In various embodiments, at least 99.1%, at least 99.2%, at least 99.3%, at least 99.4%, at least 99.5%, at least 99.6%, at least 99.7%, at least 99.8%, or at least 99.9% of the generated small molecule compounds are chemically valid. In various embodiments, at least 85% of the generated small molecule compounds are novel. In various embodiments, at least 90% of the generated small molecule compounds are novel. In various embodiments, at least 91%, at least 92%, at least 93%, at least 94%, at least 95%, at least 96%, at least 97%, at least 98%, or at least 99% of the small molecule compounds are novel. In various embodiments, the trained machine learning model is a trained neural network.

Additionally disclosed herein is a non-transitory computer readable medium comprising instructions that, when executed by a processor, cause the processor to: obtain a graph adjacency tensor, wherein the adjacency tensor comprises a representation of a small molecule compound; iteratively apply a trained machine learning model to generate the small molecule compound, wherein each iteration comprises: analyzing, using the trained machine learning model, the graph adjacency tensor to generate probabilities corresponding to available actions, wherein each probability indicates a likelihood of generating a valid substructure of a small molecule compound if a corresponding available action were taken; selecting one of the available actions based on the probabilities; updating the graph adjacency tensor with a value indicative of the selected action to generate an updated graph adjacency tensor, wherein the trained machine learning model is trained using training examples indicating actions at individual steps of the small molecule building process.

In various embodiments, the available actions comprise adding an atom, adding a bond, or assigning a charge to an atom. In various embodiments, the iterative application of the trained machine learning model terminates after the small molecule compound comprises at least a threshold number of atoms. In various embodiments, the threshold number of atoms is at least 5, at least 10, at least 20, at least 30, at least 40, or at least 50 atoms. In various embodiments, selecting one of the available actions based on the probabilities comprises selecting an available action corresponding to a highest probability. In various embodiments, the selected action comprises adding an atom, and wherein updating the graph adjacency tensor with a value indicative of the selected action comprises updating a diagonal of the graph adjacency tensor with a value indicative of the added atom. In various embodiments, the selected action comprises adding a bond, and wherein updating the graph adjacency tensor with a value indicative of the selected action comprises updating an upper portion of the graph adjacency tensor with a value indicative of the added bond. In various embodiments, the selected action comprises assigning a charge to an atom, and wherein updating the graph adjacency tensor with a value indicative of the selected action comprises updating a diagonal of the graph adjacency tensor with a value indicative of the assigned charge.

In various embodiments, the graph adjacency tensor comprises m×n×d dimensions. In various embodiments, m or n represents a pre-determined maximum number of atoms of the small molecule compound. In various embodiments, m is between 20 and 60. In various embodiments, n is between 20 and 60. In various embodiments, d is between 20 and 30. In various embodiments, analyzing, using the trained machine learning model, the graph adjacency tensor to generate probabilities corresponding to available actions further comprises: determining a subgraph of the graph adjacency tensor, the subgraph representing a substructure of the small molecule compound; and analyzing the subgraph using the trained machine learning model to predict probabilities corresponding to available actions. In various embodiments, each of the dimensions p×q×r of the subgraph are smaller than or equal to each of corresponding dimensions m×n×d of the graph adjacency tensor. In various embodiments, p is between 1 and 5. In various embodiments, q is between 1 and 5. In various embodiments, r is between 1 and 5.

In various embodiments, the training examples used to train the machine learning model are generated by: obtaining a plurality of small molecule compounds; for each of one or more small molecule compounds of the plurality, generating a plurality of subgraph-action pairs for the small molecule compound by: decomposing the small molecule compound into a sequence of actions for generating the small molecule compound; generating graph adjacency tensor subgraphs for actions in the sequence of actions; and pairing each action with a corresponding graph adjacency tensor subgraph; randomly shuffling subgraph-action pairs to break autocorrelations; and assigning randomly shuffled subgraph-action pairs to training examples. In various embodiments, the trained machine learning model is trained using at least 1 million training examples. In various embodiments, the trained machine learning model is trained using at least 10 million training examples. In various embodiments, the trained machine learning model is trained using at least 100 million training examples. In various embodiments, obtaining a plurality of small molecule compounds comprises obtaining the plurality of small molecule compounds from a publicly available dataset. In various embodiments, the publicly available dataset is any of a QM9, ZINC250k, or ChEMBL dataset. In various embodiments, at least 90% of the generated small molecule compounds are chemically valid. In various embodiments, at least 95%, at least 96%, at least 97%, at least 98%, or at least 99% of the generated small molecule compounds are chemically valid. In various embodiments, at least 99.1%, at least 99.2%, at least 99.3%, at least 99.4%, at least 99.5%, at least 99.6%, at least 99.7%, at least 99.8%, or at least 99.9% of the generated small molecule compounds are chemically valid. In various embodiments, at least 85% of the generated small molecule compounds are novel. In various embodiments, at least 90% of the generated small molecule compounds are novel. In various embodiments, at least 91%, at least 92%, at least 93%, at least 94%, at least 95%, at least 96%, at least 97%, at least 98%, or at least 99% of the small molecule compounds are novel. In various embodiments, the trained machine learning model is a trained neural network.

### BRIEF DESCRIPTION OF THE DRAWINGS

These and other features, aspects, and advantages of the present invention will become better understood with regard to the following description and accompanying drawings. It is noted that wherever practicable similar or like reference numbers may be used in the figures and may indicate similar or like functionality. For example, a letter after a reference numeral, such as “third party entity 110A,” indicates that the text refers specifically to the element having that particular reference numeral. A reference numeral in the text without a following letter, such as “third party entity 110,” refers to any or all of the elements in the figures bearing that reference numeral (e.g. “third party entity 110” in the text refers to reference numerals “third party entity 110A” and/or “third party entity 110B” in the figures).

FIG. 1A depicts an overall system environment including an in silico drug discovery system 130, in accordance with an embodiment.

FIG. 1B depicts a block diagram of the in silico drug discovery system, in accordance with an embodiment.

FIG. 2A depicts an example flow process for generating a small molecule compound, in accordance with an embodiment.

FIG. 2B depicts an example process involving the deployment of a trained machine learning model, in accordance with an embodiment.

FIG. 3A depicts an example flow process for generating training examples for use in training a machine learning model, in accordance with an embodiment.

FIG. 3B depicts an example process for generating training examples, in accordance with an embodiment.

FIG. 4 illustrates an example computing device for implementing system and methods described in FIGS. 1A, 1B, 2A, 2B, 3A, and 3B.

FIG. 5 shows an example adjacency tensor for use in generating anew molecule.

FIG. 6 shows an example step-by-step process for generating a new molecule using the adjacency tensor.

FIG. 7 shows the log frequency of atom types, charge types, and number of atoms per molecule in the ChEMBL dataset.

### DETAILED DESCRIPTION OF THE INVENTION

### Definitions

Terms used in the claims and specification are defined as set forth below unless otherwise specified.

The phrase “obtaining a graph adjacency tensor” comprises obtaining a graph adjacency tensor (e.g., from a third party or from a stored memory location), and further encompasses initializing or generating a graph adjacency tensor.

The phrase “graph adjacency tensor” refers to a representation of a small molecule compound. For example, the graph adjacency tensor can be a representation comprising one or more of an atom, bond, charge, or valence of a small molecule compound. In various embodiments, the graph adjacency tensor is a representation of an incomplete small molecule compound, such as a small molecule compound that is in the process of being generated. In such embodiments, the graph adjacency tensor of an incomplete small molecule compound is a representation including atoms, bonds, charges, or valences of the incomplete small molecule compound.

The phrase “analyzing, using the trained neural network, the graph adjacency tensor” encompasses analyzing an obtained graph adjacency tensor (e.g., obtained from a third party) and further encompasses analyzing an updated graph adjacency tensor (e.g., updated from a prior iteration of the small molecule compound generation process).

The phrase “obtaining a compound” comprises physically obtaining a compound. “Obtaining a compound” also encompasses obtaining a representation of the compound. Examples of a representation of the compound include a molecular representation such as a molecular fingerprint or a molecular graph. “Obtaining a compound” also encompasses obtaining the compound expressed as a particular structure format. Example structure formats of the compound include any of a simplified molecular-input line-entry system (SMILES) string, MDL MOL, SDF, PDB, xyz, inchi, and mol2 format.

As used herein, a “subgraph” refers to portion of a graph adjacency tensor that corresponds to a substructure of a small molecule compound. As one example, a substructure of the small molecule compound refers to an intermediate small molecule compound that is in the generation process. As another example, a substructure of the small molecule compound refers to a portion of the small molecule compound corresponding to a decomposed graph adjacency tensor (e.g., decomposition of a graph adjacency tensor to generate subgraph-action pairs for inclusion in training data).

It must be noted that, as used in the specification and the appended claims, the singular forms “a,” “an” and “the” include plural referents unless the context clearly dictates otherwise.

System Overview

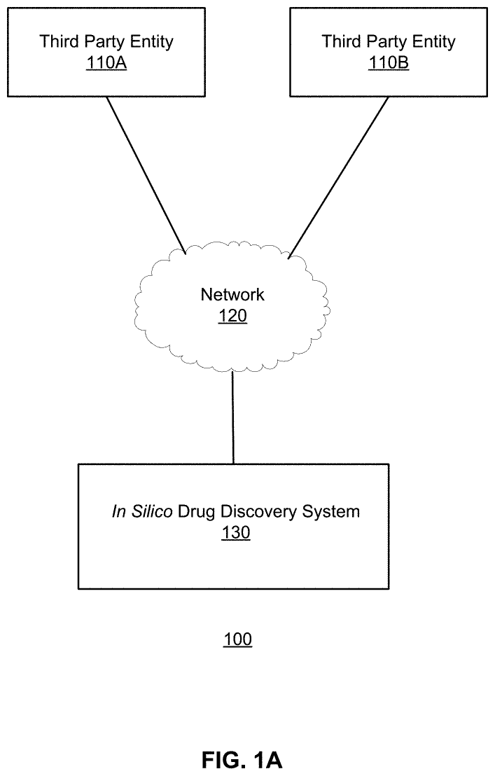

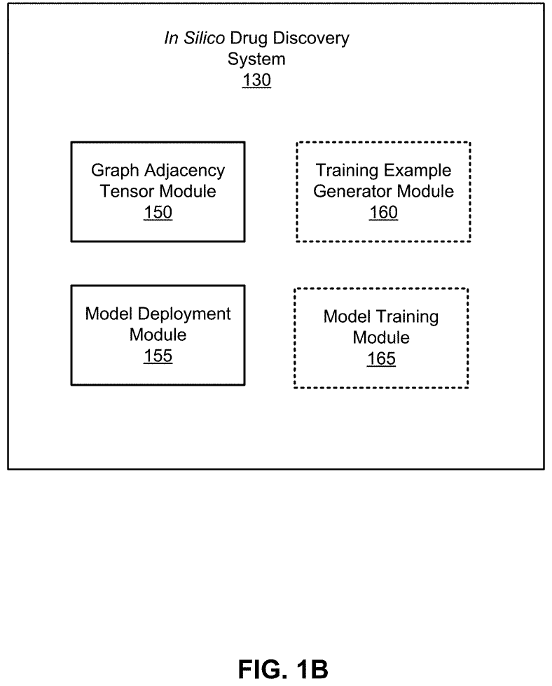

FIG. 1A depicts an overall system environment 100 including an in silico drug discovery system 130, in accordance with an embodiment. FIG. 1A further introduces one or more third party entities 110A and 110B in communication with one another and/or the in silico drug discovery system through a network 120. FIG. 1A depicts one embodiment of the overall system environment 100. In other embodiments, additional or fewer third party entities 110 in communication with the in silico drug discovery system 130 can be included.

Generally, the in silico drug discovery system 130 performs methods disclosed herein, such as methods for discovering valid small molecule compounds. Such small molecule compounds may not have been previously developed and represent novel compounds that may be useful for various purposes (e.g., treating disease). The third party entities 110 communicate with the in silico drug discovery system 130 for purposes associated with discovering valid small molecule compounds.

In various embodiments, the methods described herein as being performed by the in silico drug discovery system 130 can be dispersed between the in silico drug discovery system 130 and third party entities 110. For example, a third party entity 110A or 110B can generate training data and/or train a machine learning model. The in silico drug discovery system 130 can then deploy the machine learning model to discover new, valid small molecule compounds.

Referring to the third party entities 110, in various embodiments, a third party entity 110 represents a partner entity of the in silico drug discovery system 130. For example, the third party entity 110 can operate either upstream or downstream of the in silico drug discovery system 130. In various embodiments, a first third party entity 110A can operate upstream of the in silico drug discovery system 130 and a second third party entity 1101B can operate downstream of the in silico drug discovery system 130.

As one example, the third party entity 110 operates upstream of the in silico drug discovery system 130. In such a scenario, the third party entity 110 may perform the methods of generating training data and training of machine learning models, as is described in further detail herein. Thus, the third party entity 110 can provide trained machine learning models to the in silico drug discovery system 130 such that the in silico drug discovery system 130 can deploy the trained machine learning models.

As another example, the third party entity 110 operates downstream of the in silico drug discovery system 130. In this scenario, the in silico drug discovery system 130 deploy trained machine learning models to discover new, valid small molecule compounds. The in silico drug discovery system 130 can provide identification of one or more discovered small molecule compounds (e.g., in an electronic format such as a simplified molecular-input line-entry system (SMILES) string) to the third party entity 110. The third party entity 110 can use the identification of the discovered small molecule compounds. In various embodiments, the third party entity 110 may be an entity that synthesizes the discovered small molecule compounds (e.g., a contract research organization (CRO) or a pharmaceutical company).

Referring to the network 120 shown in FIG. 1A, this disclosure contemplates any suitable network 120 that enables connection between the in silico drug discovery system 130 and third party entities 110. The network 120 may comprise any combination of local area and/or wide area networks, using both wired and/or wireless communication systems. In one embodiment, the network 120 uses standard communications technologies and/or protocols. For example, the network 120 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 120 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 120 may be represented using any suitable format, such as hypertext markup language (HTML) or extensible markup language (XML). In some embodiments, all or some of the communication links of the network 120 may be encrypted using any suitable technique or techniques.

FIG. 1B depicts a block diagram of the in silico drug discovery system 130, in accordance with an embodiment. FIG. 1B introduces individual modules of the in silico drug discovery system 130 which includes, in various embodiments, a graph adjacency tensor module 150, a model deployment module 155, a training example generator module 160, and a model training module 165. Generally, the graph adjacency tensor module 150 manages graph adjacency tensors (e.g., generates and/or updates graph adjacency tensors). For example, when in the process of generating a new small molecule compound, the graph adjacency tensor module 150 sequentially updates the graph adjacency tensor to reflect the added atoms, bonds, and/or charges. The model deployment module 155 deploys a trained machine learning model to predict and select an action (e.g., adding a bond, charge, or atom). Thus, the selected action can be undertaken to generate a new small molecule compound. The model deployment module 155 can deploy a trained machine learning model across multiple iterations, thereby selecting and undertaking an action to further generate an updated graph adjacency tensor module representing the new small molecule compound. The training example generator module 160 generates training data for use in training machine learning models. In particular embodiments, the training example generator module 160 generates training data which enables the training of the machine learning model on different variations of decomposed training molecules. The model training module 165 trains one or more machine learning models using the training data. Generally, by training models on training data disclosed herein, this ensures that the trained machine learning model predicts an action that, if taken, would lead to a new, valid subgraph (as opposed to a full molecule) that incorporates the action. Thus, in comparison to conventional strategies, such trained machine learning models disclosed herein predict new small molecule compounds with higher rates of validity.

In various embodiments, the in silico drug discovery system 130 may be differently configured than as shown in FIG. 1B. For example, there may be additional or fewer modules in the in silico drug discovery system 130 than shown in FIG. 1B. In various embodiments, the in silico drug discovery system 130 need not include the training example generator module 160 or the model training module 165. In such embodiments, the steps of generating training examples (performed by the training example generator module 160) and/or training machine learning models (performed by the model training module 165) can be performed by a third party, and then the trained machine learning models can be provided to the in silico drug discovery system 130. Further details of the particular methods performed by the graph adjacency tensor module 150, model deployment module 155, training example generator module 160, and model training module 165 are described in further detail herein.

Example Methods

Example Flow Process for Generating New Molecules

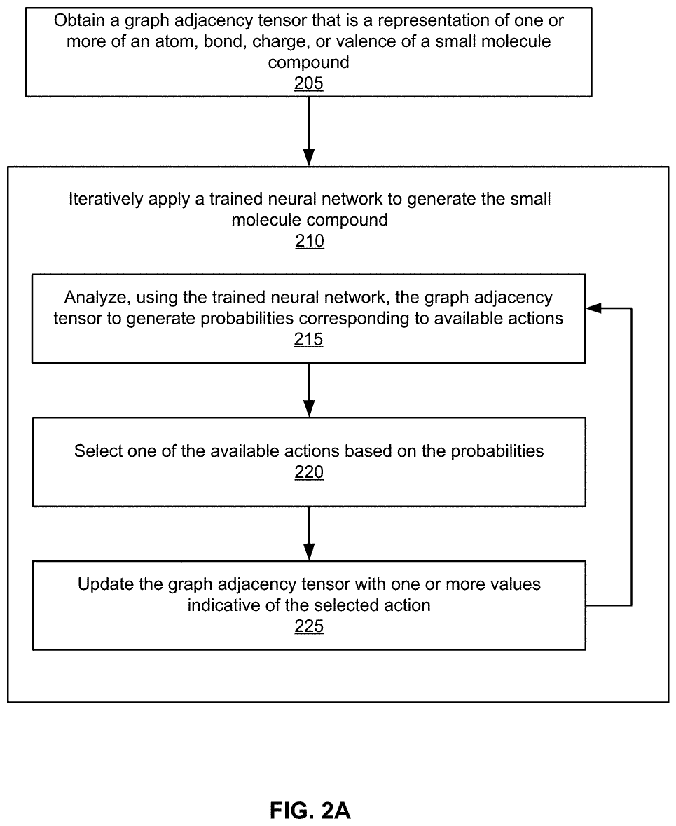

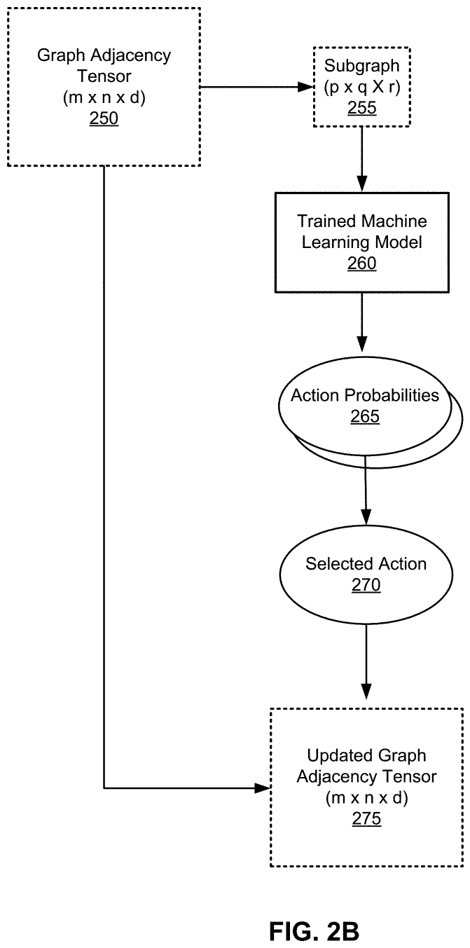

Disclosed herein is a machine learning-based sequential graph generation algorithm for generating new small molecule compounds. In various embodiments, such a machine learning-based sequential graph generation algorithm involves implementation of a machine learning model for sequentially generating a new and valid small molecule compound. Reference is made to FIG. 2A, which depicts an example flow process for generating a small molecule compound, in accordance with an embodiment. The flow process of FIG. 2A shows steps 205, 210, 215, 220, and 225. Here, steps 215, 220, and 225 are iteratively performed, where each iteration is represented by step 210. In various embodiments, the flow process for generating a small molecule compound may include additional or fewer steps than shown in the flow process of FIG. 2A. Further reference will be made to FIG. 2B which depicts an example process involving the deployment of a trained machine learning model, in accordance with an embodiment.

Beginning at step 205 in FIG. 2A, the process begins by obtaining a graph adjacency tensor. Generally, the step of obtaining the graph adjacency tensor is performed by the graph adjacency tensor module 150 (as shown in FIG. 1B). In various embodiments, the step of obtaining the graph adjacency tensor includes generating the graph adjacency tensor based on a small molecule compound. In various embodiments, the step of obtaining the graph adjacency tensor includes receiving the graph adjacency tensor (e.g., from a third party that generated the graph adjacency tensor).

The graph adjacency tensor comprises a representation of one or more of an atom, bond, charge, or valence of a small molecule compound. In various embodiments, the graph adjacency tensor is a representation of each of the atom, bond, charge, and valence of a small molecule compound. In various embodiments, the graph adjacency tensor is a representation of an incomplete small molecule compound, such as a small molecule compound that is in the process of being generated. For example, upon initialization for generating a new small molecule compound, the graph adjacency tensor may be empty, thereby representing a first stage of a completely new small molecule compound (with no atoms, bonds, charge). As another example, the incomplete small molecule compound may include one or more atoms, bonds, or charges and thus, the graph adjacency tensor is a representation of the atoms, bonds, and charges of the incomplete small molecule compound. The graph adjacency tensor representing the incomplete small molecule can then be further iteratively updated to incorporate additional atoms, bonds, and charges until the small molecule compound is complete.

Referring to FIG. 2B, the graph adjacency tensor 250 may have dimensions of m×n×d. In various embodiments, the dimensions of the graph adjacency tensor 250 are fixed irrespective of the compound generation process. For example, upon initialization for generating a new small molecule compound, the graph adjacency tensor may be empty but may still have dimensions of m×n×d. In some embodiments, the dimensions of the graph adjacency tensor 250 are variable depending on the steps of the compound generation process. For example, the graph adjacency tensor may have smaller dimensions at an earlier stage of the compound generation process in comparison to the graph adjacency tensor at a later stage of the compound generation process. Further details of the graph adjacency tensor 250 are described herein.

In various embodiments, as shown in FIG. 2B, the graph adjacency tensor 250 may include a subgraph 255. The subgraph may have dimensions p×q×r. Generally, the subgraph 255 refers to portion of a graph adjacency tensor 250 that corresponds to a substructure of a small molecule compound. For example, the subgraph 255 may refer to the portion of the graph adjacency tensor 250 corresponding to a substructure for which a bond, charge, or atom is to be added. Thus, each of the dimensions of the subgraph 255 are smaller than, or equal to, the dimensions of the graph adjacency tensor 250. For example, the dimension p of the subgraph 255 is less than or equal to the dimension m of the graph adjacency tensor 250. As another example, the dimension q of the subgraph 255 is less than or equal to the dimension n of the graph adjacency tensor 250. As another example, the dimension r of the subgraph 255 is less than or equal to the dimension d of the graph adjacency tensor 250. Further details of the subgraph 255 are described herein.

Returning to FIG. 2A, step 210 involves iteratively applying a trained machine learning model to generate the small molecule compound. Each iteration involves step 215 (to generate probabilities of an available action using a machine learning model), step 220 (to select one of the available actions based on the probabilities), and step 225 (to update the graph adjacency tensor with values indicative of the selected action). Generally, step 210 can be performed by the model deployment module 155, as described in FIG. 1B.

Specifically, step 215 involves analyzing, using the trained machine learning model, the graph adjacency tensor to generate probabilities corresponding to available actions, wherein each probability indicates a likelihood of generating a new valid subgraph given a corresponding available action. For example, after X iterations involving the application of the machine learning model, X actions (e.g., adding a bond, charge, or atom) may have been taken, leading to an intermediate small molecule compound or a substructure of a small molecule compound made up of the bonds, charges, and/or atoms of the X actions. In various embodiments, a substructure refers to at least a threshold number of atoms connected to each other that make up the intermediate small molecule compound. In some embodiments, the threshold number may be 1 atom of an intermediate small molecule compound. In some embodiments, the threshold number may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 atoms of an intermediate small molecule compound.

Examples of available actions include, but are not limited to: adding an atom type (e.g., hydrogen, helium, lithium, beryllium, boron, carbon, nitrogen, oxygen, fluorine, neon, sodium, magnesium, aluminum, silicon, phosphorous, sulfur, chlorine, argon, or potassium atoms), adding a bond type (e.g., no bond, single bond, double bond, triple bond), or assigning a charge to an atom. In various embodiments, an available action is limited by the atoms, bonds, or charges that are currently present in the graph adjacency tensor. For example, a bond adding action is only allowed between a new atom and other existing atoms. The addition or modification of bonds between existing atoms are not permitted.

In various embodiments, the trained machine learning model is configured to receive, as input, a graph adjacency tensor. The trained machine learning model analyzes the graph adjacency tensor and outputs a range of probabilities for available actions. For each available action, the trained machine learning model generates a probability for that available action. Each probability represents a likelihood that the corresponding available action, if taken, will result in generating a new valid subgraph corresponding to a substructure of a small molecule compound.

In various embodiments, as shown in FIG. 2B, the trained machine learning model 260 is configured to receive, as input, a subgraph 255 of a graph adjacency tensor 250. The trained machine learning model analyzes the subgraph 255 of the graph adjacency tensor 250 and outputs a range of action probabilities 265 for available actions. Each probability represents a likelihood that the corresponding available action, if taken, will result in generating a new valid subgraph corresponding to a substructure of a small molecule compound.

There is an obstacle to be overcome to enable scalable pretraining for larger molecular graphs. As the number of atoms increases in a molecular graph, the size of its corresponding adjacency tensor grows quadratically. This also leads to quadratic increase in the size of the action space, as the number of possible bonding choices between atoms also grow quadratically with the number of atoms. If not restricted properly, similar model collapse issue of one-shot graph generations will be encountered.

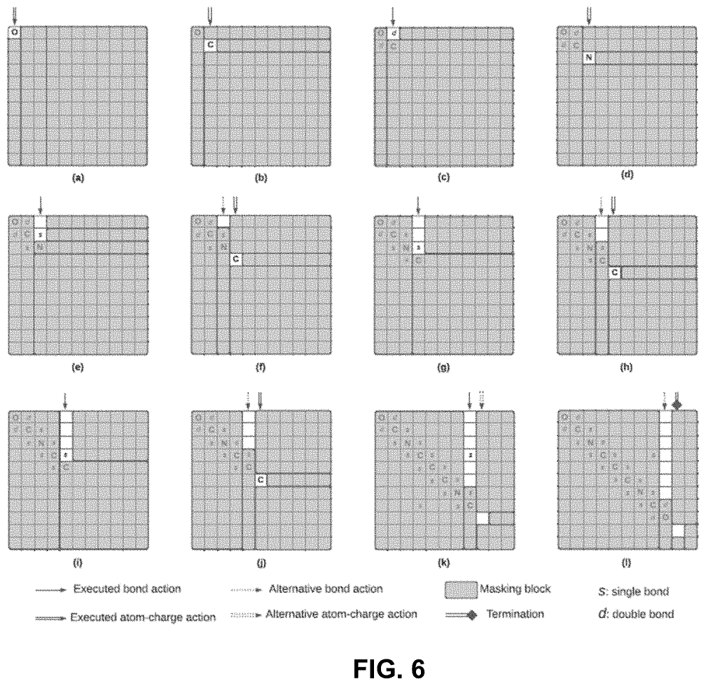

Disclosed herein is a simple restriction mechanism to curb the growth of the action space as illustrated in FIG. 6. At each iteration, possible actions are restricted (e.g., only actions allowed in a single column with unmasked cells). Specifically, bond adding action is only allowed between the new atom and other existing atoms; the addition or modification of bonds between existing atoms are not permitted. Thus, the machine learning model need only determine probabilities for a limited number of possible actions.

In various embodiments, to further restrict the action space, atom and charge creations are only allowed in the diagonal section of the graph adjacency tensor, whereas bond creations are only allowed in the upper triangular section of the graph adjacency tensor, and then are mirrored across the diagonal. These restrictions together limit the action space growth from an unrestricted quadratic one to a linear one with respect to the number of atoms. For example, the maximum size of the action space for creating a molecular graph with a number of atoms, t atom types having c charge types, and b bond types will be;

d_{action}=t×c+(a−1)×b (1)

Returning to FIG. 2A, step 220 involves selecting one of the available actions based on the probabilities (e.g., shown as selected action 270 in FIG. 2B). In various embodiments, the action is sampled from the probabilities, such that actions corresponding to higher probabilities are more likely to be selected than actions corresponding to lower probabilities. In such embodiments, the available action with the highest probability need not always be selected. In various embodiments, the available action corresponding to the highest probability selected. For example, given that the probability represents a likelihood that the corresponding available action, if taken, will result in generating a valid subgraph, selecting the available action with the highest probability will maximize the likelihood that the final small molecule compound is valid.

Step 225 involves updating the graph adjacency tensor with one or more values indicative of the selected action to generate a graph adjacency tensor for analysis in a next iteration. For example, if the selected action is the addition of an atom type (e.g., hydrogen, helium, lithium, beryllium, boron, carbon, nitrogen, oxygen, fluorine, neon, sodium, magnesium, aluminum, silicon, phosphorous, sulfur, chlorine, argon, or potassium), the graph adjacency tensor is updated with a value to reflect the addition of the selected atom type. In various embodiments, the addition of an atom involves updating the diagonal of the graph adjacency tensor with a representation of the added atom. As another example, if the selected action is the addition of a particular bond type (e.g., no bond, single bond, double bond, triple bond), the graph adjacency tensor is updated with a value to reflect the addition of the selected bond type. In various embodiments, the addition of a bond involves updating one of an upper portion or bottom portion of the graph adjacency tensor. As another example, if the selected action is the assigning of a charge, the graph adjacency tensor is updated with a value to reflect the charge assignment. In various embodiments, the assignment of a charge to an atom involves updating an atom along the diagonal of the graph adjacency tensor.

As shown in FIG. 2B, the updated graph adjacency tensor 275 may be generated by taking the selected action 270 (e.g., one of adding a bond, charge, or atom) on the original graph adjacency tensor 250. Here, the dimensions of the original graph adjacency tensor 250 (e.g., m×n×d) may not differ from the dimensions of the updated graph adjacency tensor 275 (e.g., also m×n×d). Rather, an entry of the updated graph adjacency tensor 275 may be populated which reflects the selection action 270.

Following generation of the updated graph adjacency tensor, if the small molecule compound is not yet completed, then further iterations of steps 215, 220, and 225 are subsequently performed. For example, in the next iteration, the updated graph adjacency tensor is analyzed by the trained machine learning model to determine the next available action that is likely to lead to a valid small molecule compound. Steps 215, 220, and 225 are iteratively performed until the small molecule compound is completed.

In various embodiments, generation of the small molecule compound will terminate if the number of atoms generated reaches a predetermined number. In various embodiments, the predetermined number of atoms is at least 5 atoms. In various embodiments, the predetermined number of atoms is at least 10 atoms. In various embodiments, the predetermined number of atoms is at least 20 atoms. In various embodiments, the predetermined number of atoms is at least 30 atoms. In various embodiments, the predetermined number of atoms is at least 40 atoms. In various embodiments, the predetermined number of atoms is at least 50 atoms. In various embodiments, the predetermined number of atoms is at least 100 atoms. In various embodiments, the predetermined number of atoms is at least 150 atoms. In various embodiments, the predetermined number of atoms is at least 200 atoms. In various embodiments, the predetermined number of atoms is at least 250 atoms. In various embodiments, the predetermined number of atoms is at least 300 atoms. In various embodiments, the predetermined number of atoms is at least 350 atoms. In various embodiments, the predetermined number of atoms is at least 400 atoms. In various embodiments, the predetermined number of atoms is at least 450 atoms. In various embodiments, the predetermined number of atoms is at least 500 atoms.

After a small molecule compound is completed, the validity of the generated small molecule compound can be determined. In various embodiments, a small molecule compound is defined as chemically valid if it can be successfully converted to a particular structure format. Example structure formats of the small molecule compound include any of a simplified molecular-input line-entry system (SMILES) string, MDL Molfile (MDL MOL), Structure Data File (SDF), Protein Data Bank (PDB), Molecule specification file (xyz), International Union of Pure and Applied Chemistry (IUPAC) International Chemical Identifier (InChI), and Tripos Mol2 file (mol2) format. In particular embodiments, a small molecule compound is chemically valid if the generated graph adjacency tensor can be converted to a canonical SMILES string using RDKit.

In various embodiments, at least 90% of the small molecule compounds generated using the process shown in FIGS. 2A and 2B are chemically valid. In various embodiments, at least 95%, at least 96%, at least 97%, at least 98%, or at least 99% of the small molecule compounds generated using the process shown in FIGS. 2A and 2B are chemically valid. In various embodiments, at least 99.1%, at least 99.2%, at least 99.3%, at least 99.4%, at least 99.5%, at least 99.6%, at least 99.7%, at least 99.8%, or at least 99.9% of the small molecule compounds generated using the process shown in FIGS. 2A and 2B are chemically valid. In various embodiments, 100% of the small molecule compounds generated using the process shown in FIGS. 2A and 2B are chemically valid.

In various embodiments, at least 85% of the small molecule compounds generated using the process shown in FIGS. 2A and 2B are novel. As used herein, “novel” small molecule compounds generated using the process shown in FIGS. 2A and 2B refer to compounds that that are not present in the training dataset. In various embodiments, at least 90% of the small molecule compounds generated using the process shown in FIGS. 2A and 2B are novel. In various embodiments, at least 91%, at least 92%, at least 93%, at least 94%, at least 95%, at least 96%, at least 97%, at least 98%, or at least 99% of the small molecule compounds generated using the process shown in FIGS. 2A and 2B are novel. In various embodiments, at least 99.1%, at least 99.2%, at least 99.3%, at least 99.4%, at least 99.5%, at least 99.6%, at least 99.7%, at least 99.8%, or at least 99.9% of the small molecule compounds generated using the process shown in FIGS. 2A and 2B are novel. In various embodiments, 100% of the small molecule compounds generated using the process shown in FIGS. 2A and 2B are novel.

Example Training of Machine Learning Models

Graph generation can be decomposed into a sequence of atom adding and bond adding actions. These actions are conditioned on the subgraph generated at the previous step. In various embodiments, the next atom and/or bond to be added is predicted by a machine learning model. In various embodiments, training of a machine learning model can be indirectly achieved by maximizing the joint likelihood of all sequential actions leading to reconstruction of graphs in the training dataset. In particular embodiments, training of a machine learning model involves a more direct way of learning to predict the correct action at each step. A training dataset for use in training a machine learning model can be obtained by decomposing the adjacency tensor into many subgraph-action pairs. Examples of subgraph-action pairs are shown in FIG. 6.

Generally, the training methodology disclosed herein ensures that the machine learning model learns from training examples at each step of the molecule generation process. In particular embodiments, the machine learning model disclosed herein is a neural network. In particular embodiments, the machine learning model is a deep learning model. For example, the training methodology disclosed herein ensures that a machine learning model is presented with training examples corresponding to an early stage of molecule generation (e.g., less than 10 atoms and bonds in a graph adjacency tensor), training examples corresponding to mid-stage of molecule generation (e.g., between 10 and 30 atoms and bonds in a graph adjacency tensor), and training examples corresponding to the late stage of molecule generation (e.g., more than 30 atoms and bonds in a graph adjacency tensor). To generate these training examples across the various stages of molecule generation, training molecules are decomposed into individual steps (e.g., atom adding or bond adding). Thus, a single training molecule can be decomposed into X different actions, which can further be assigned to X different training examples. Thus, by training the machine learning model using these training examples, the machine learning model can learn appropriate actions to be taken at different stages of the molecule generation process.

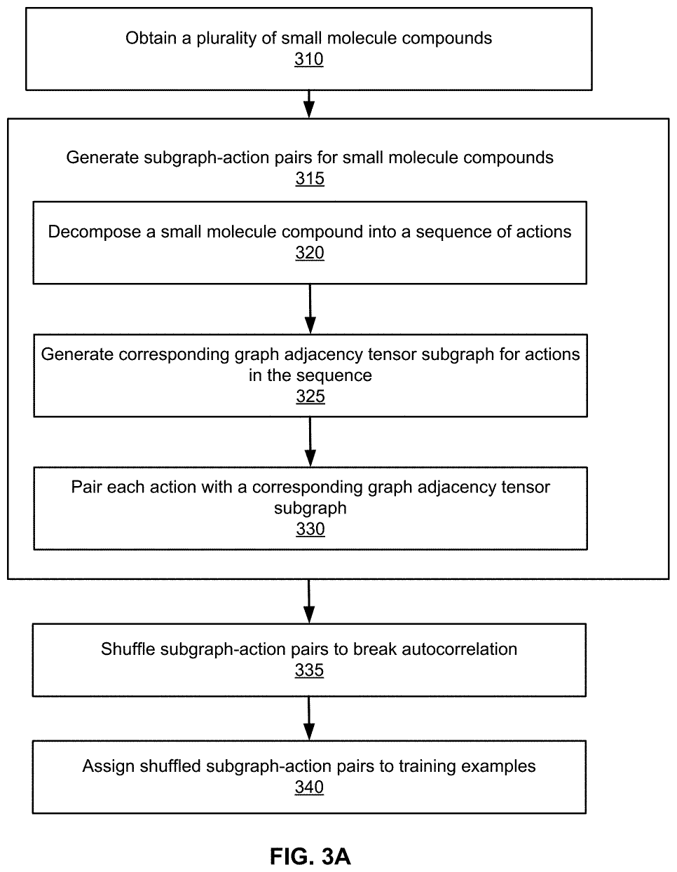

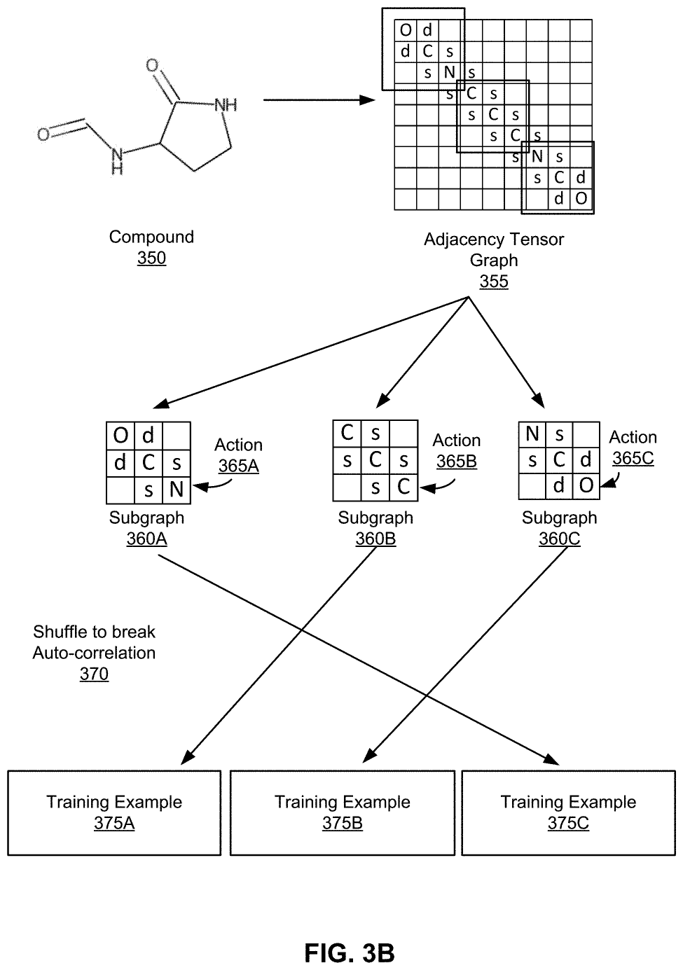

FIG. 3A depicts an example flow process for generating training examples for use in training a machine learning model, in accordance with an embodiment. Further reference will be made to FIG. 3B which depicts an example process for generating training examples, in accordance with an embodiment.

As shown in FIG. 3A, step 310 involves obtaining a plurality of small molecule compounds. For simplicity purposes, FIG. 3B shows a single example of a small molecule compound 350. In various embodiments, small molecule compounds are obtained as a representation, such as a molecular fingerprint or a molecular graph of the small molecule compound. In various embodiments, these small molecule compounds that are used to generate training examples, also referred to as training compounds or training molecules, are obtained from a dataset. A dataset may be a publicly available dataset, such as any one of a QM9, ZINC250k, or ChEMBL dataset.

Step 315 involves generating subgraph-action pairs for the small molecule compounds. Step 315 may involve each of steps 320, 325, and 330. Step 320 involves decomposing a small molecule compound into a sequence of actions. For example, the sequence of actions can include the addition of atoms, bonds, and/or charges beginning from an empty graph adjacency tensor to a graph adjacency tensor corresponding to the full small molecule compound. Referring to FIG. 3B, it shows the corresponding adjacency tensor graph 355 of the compound 350. Here, atoms (denoted as “O” for oxygen, “C” for carbon, and “N” for nitrogen) of the compound 350 are situated along the diagonal of the adjacency tensor graph 355, whereas the bonds (denoted as “d” for a double bond and “s” for a single bond) are shown in entries adjacent to the entries of the diagonal.

Step 315 may involve decomposing actions, such as an addition of a bond or the addition of an atom, into a sequence. For example, referring to the adjacency tensor graph 355, the top left entry of an “O” oxygen atom may be a first action followed by the action of a double bond denoted as “d” in the entries adjacent to the “O” oxygen atom. A subsequent action can include the entry of a “C” carbon atom following along the diagonal of the adjacency tensor graph 355, and so forth.

Returning to FIG. 3A, step 325 involves generating corresponding graph adjacency tensor subgraphs for each action in the sequence. Generally, a graph adjacency tensor subgraph is a portion of the graph adjacency tensor. For example, a graph adjacency tensor subgraph encompasses a threshold number of atoms and/or bonds in a small molecule compound. In such embodiments, this enables a machine learning model to learn the appropriate action based on atoms and bonds of the subgraph.

For purposes of simplicity, FIG. 3B shows three example subgraphs (e.g., subgraph 360A, subgraph 360B, and subgraph 360C) that are generated from the adjacency tensor graph 355. In practice, the adjacency tensor graph 355 may have additional corresponding subgraphs. Here, each of subgraph 360A, subgraph 360B, and subgraph 360C have dimensions p×q×r, where p=3, q=3, and r=1. However, in other embodiments, the dimensions of the subgraphs may differ.

Returning to FIG. 3A, at step 330, each action is paired with a corresponding graph adjacency tensor subgraph. In various embodiments, for an action that is paired with a corresponding subgraph, the action refers to the last taken action in the subgraph. As an example, FIG. 3B shows that subgraph 360A may be paired with a corresponding action 365A. Here, the action 365A may refer to the most recent action involving the addition of the “N” nitrogen atom to the subgraph 360A. As another example, FIG. 3B shows that subgraph 360B may be paired with a corresponding action 365B. Here, the action 365B may refer to the most recent action involving the addition of the “C” carbon atom to the subgraph 360B. As another example, FIG. 3B shows that subgraph 360C may be paired with a corresponding action 365C. Here, the action 365C may refer to the most recent action involving the addition of the “0” oxygen atom to the subgraph 360C. Thus, this completes step 315 of generating subgraph-action pairs for a small molecule compound.

Returning to FIG. 3A, at step 335, the subgraph-action pairs are randomly shuffled to break their autocorrelation. For example, FIG. 3B shows the shuffling 370 of subgraph-action pairs to break autocorrelation. In various embodiments, subgraph-action pairs can be shuffled across various small molecule compounds (as opposed to the single compound 350 shown in FIG. 3B). By shuffling the subgraph-action pairs, this ensures that the training of a machine learning model occurs independent of the particular small molecule compound that a pairing originates from.

At step 340, the shuffled subgraph-action pairs are assigned to training examples. For example, in FIG. 3B, the pairing of the subgraph 360B and action 365B may be assigned to a first training example 375A. Additionally, the pairing of the subgraph 360C and action 365C may be assigned to a second training example 375B. Furthermore, the pairing of the subgraph 360A and action 365A may be assigned to a third training example 375C. Thus, these training examples include subgraph-action pairs that are independent of other subgraph-action pairs.

In various embodiments, the action of a subgraph-action pair in a training example serves as a ground truth. In various embodiments, the action can undergo one-hot encoding. Thus, the machine learning model can be trained using the training examples. In various embodiments, the machine learning model is trained using a “teacher forcing” strategy; that is, the correct action is always executed to generate subgraph regardless of the predicted action of a generative model.

Example Graph Adjacency Tensor

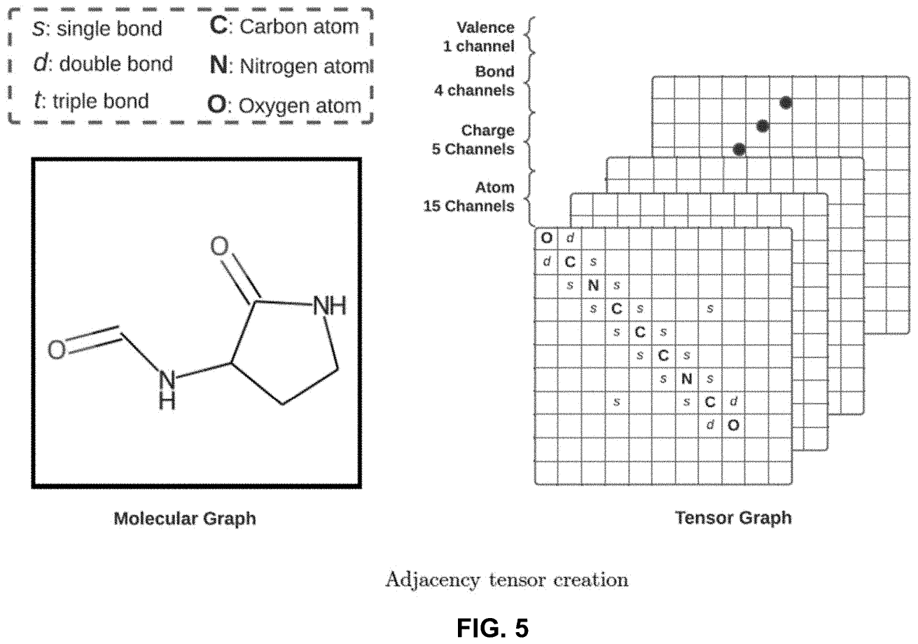

A graph adjacency tensor represents a molecular graph of a chemical compound with a cuboid. Atoms features are placed along the diagonal of the adjacency tensor. These features include atom type, atom charge and the remaining valence. On the other hand, connection features are placed in the off-diagonal section of the tensor, and are mirrored across the diagonal to ensure symmetry. An example of the adjacency tensor is shown in FIG. 5.

In various embodiments, the feature channel of the adjacency tensor is a stack of 4 smaller channel groups, which are the atom channels, charge channels, bond channels, and the valence channel. All channels are one-hot encoded except for the valence channel which contains an integer indicating the remaining valence of an atom. This remaining valence may be interpreted as the number of remaining single bonds an atom can establish explicitly with another non-hydrogen atom or implicitly with a hydrogen atom. As the number of bonds an atom forms with others increases, its remaining valence decreases. However, the remaining valence of any atom may not be less than 1 in a valid molecular graph. Incorporation of remaining valence as a feature enables a machine learning model to learn the relationship between a valid bond and the remaining valence. In addition, it also allows easy masking of invalid bond adding action during prediction to further improve validity rate.

In various embodiments, a graph adjacency tensor has a size of m×n×d dimensions. In various embodiments, the size of the graph adjacency tensor is fixed across various implementations such that it can adequately represent various small molecule compounds. In various embodiments, m and n can be selected to adequately represent the number of atoms in a small molecule compound. In various embodiments, m is equal to n.

In various embodiments, m is greater than 10. In various embodiments, m is greater than 20. In various embodiments, m is greater than 25. In various embodiments, m is greater than 30. In various embodiments, m is greater than 35. In various embodiments, m is greater than 40. In various embodiments, m is greater than 45. In various embodiments, m is greater than 50. In various embodiments, m is greater than 75. In various embodiments, m is greater than 100. In various embodiments, m is greater than 150. In various embodiments, m is greater than 200. In various embodiments, m is greater than 300. In various embodiments, m is greater than 400. In various embodiments, m is greater than 500. In various embodiments, m is between 10 and 100. In various embodiments, m is between 20 and 60. In various embodiments, m is between 30 and 50. In various embodiments, m is between 35 and 45. In various embodiments, m is between 38 and 42.

In various embodiments, n is greater than 10. In various embodiments, n is greater than 20. In various embodiments, n is greater than 25. In various embodiments, n is greater than 30. In various embodiments, n is greater than 35. In various embodiments, n is greater than 40. In various embodiments, n is greater than 45. In various embodiments, n is greater than 50. In various embodiments, n is greater than 75. In various embodiments, n is greater than 100. In various embodiments, n is greater than 150. In various embodiments, n is greater than 200. In various embodiments, n is greater than 300. In various embodiments, n is greater than 400. In various embodiments, n is greater than 500. In various embodiments, n is between 10 and 100. In various embodiments, n is between 20 and 60. In various embodiments, n is between 30 and 50. In various embodiments, n is between 35 and 45. In various embodiments, n is between 38 and 42.

Generally, the dimension d refers to the number of channels of the graph adjacency tensor. In various embodiments, d is one channel. In various embodiments, d is 2. In various embodiments, d is 3. In various embodiments, d is 4. In various embodiments, d is 5. In various embodiments, d is greater than 5. In various embodiments, d is greater than 10. In various embodiments, d is greater than 15. In various embodiments, d is greater than 20. In various embodiments, d is greater than 25. In various embodiments, d is greater than 30. In various embodiments, d is greater than 35. In various embodiments, d is greater than 40. In various embodiments, d is greater than 45. In various embodiments, d is greater than 50. In various embodiments, d is between 10 and 50. In various embodiments, d is between 15 and 40. In various embodiments, d is between 20 and 30. In various embodiments, d is between 22 and 26.

In various embodiments, each of the channels represents one of atoms, bonds, charges, or remaining valence of the small molecule compound. In various embodiments, at least 5 channels represent atoms of the small molecule compound. In various embodiments, at least 10 channels represent atoms of the small molecule compound. In various embodiments, at least 15 channels represent atoms of the small molecule compound. In various embodiments, at least 3 channels represent charges of the small molecule compound. In various embodiments, at least 5 channels represent charges of the small molecule compound. In various embodiments, at least 8 channels represent charges of the small molecule compound. In various embodiments, at least 10 channels represent charges of the small molecule compound. In various embodiments, at least 2 channels represent bonds of the small molecule compound. In various embodiments, at least 4 channels represent bonds of the small molecule compound. In various embodiments, at least 7 channels represent bonds of the small molecule compound. In various embodiments, at least 10 channels represent bonds of the small molecule compound. In various embodiments, at least 1 channel represent remaining valence of the small molecule compound. In various embodiments, at least 2 channels represent remaining valence of the small molecule compound. In particular embodiments, a graph adjacency tensor includes the following channels: 15 atom channels, 5 charge channels, 4 bond channels, and 1 valence channel. Examples of the graph adjacency tensor are described herein e.g., in Example 1 and FIGS. 5 and 6.

In particular embodiments, the graph adjacency tensor has a size of m×n×d. In various embodiments, values of m and n are determined according to the maximum number of atoms of the small molecule compound and d is the depth of feature channels.

In particular embodiments, the feature channels d comprise four smaller blocks, including;
One-hot encoded atom block indicating the presence of an atom type (C, N, O, etc.);
One-hot encoded charge block indicating atom charge type (−1, 0, +1, etc.);
One-hot encoded bond block indicating bond type (No bond, Single, Double, Triple);
Single integer channel representing the remaining valence of a connected atom.

As described herein the graph adjacency tensor may include a subgraph, representing the graph adjacency tensor corresponding to a substructure of a small molecule compound. In various embodiments, the graph adjacency tensor may include various subgraphs, wherein the dimensions of each of the subgraphs is less than or equal to the dimensions of the graph adjacency tensor. For example, the graph adjacency tensor may have a size of m×n×d, whereas a subgraph may have dimensions of p×q×r, where p≤m, q≤n, and r≤d. In various embodiments, the dimension p or the subgraph may equal the dimension q of the subgraph.

In various embodiments, p is less than 100. In various embodiments, p is less than 50. In various embodiments, p is less than 25. In various embodiments, p is less than 20. In various embodiments, p is less than 15. In various embodiments, p is less than 12. In various embodiments, p is less than 10. In various embodiments, p is less than 9. In various embodiments, p is less than 8. In various embodiments, p is less than 7. In various embodiments, p is less than 6. In various embodiments, p is less than 5. In various embodiments, p is less than 4. In various embodiments, p is less than 3. In various embodiments, p is between 1 and 10. In various embodiments, p is between 1 and 5. In various embodiments, p is between 1 and 3. In various embodiments, p is 2. In various embodiments, p is 3. In various embodiments, p is 4. In various embodiments, p is 5.

In various embodiments, q is less than 100. In various embodiments, q is less than 50. In various embodiments, q is less than 25. In various embodiments, q is less than 20. In various embodiments, q is less than 15. In various embodiments, q is less than 12. In various embodiments, q is less than 10. In various embodiments, q is less than 9. In various embodiments, q is less than 8. In various embodiments, q is less than 7. In various embodiments, q is less than 6. In various embodiments, q is less than 5. In various embodiments, q is less than 4. In various embodiments, q is less than 3. In various embodiments, q is between 1 and 10. In various embodiments, q is between 1 and 5. In various embodiments, q is between 1 and 3. In various embodiments, q is 2. In various embodiments, q is 3. In various embodiments, q is 4. In various embodiments, q is 5.

Generally, the dimension r refers to the number of channels of the subgraph. In various embodiments, r is one channel. In various embodiments, r is 2. In various embodiments, r is 3. In various embodiments, r is 4. In various embodiments, r is 5. In various embodiments, r is between 1 and 5. In various embodiments, r is greater than 5. In various embodiments, r is greater than 10. In various embodiments, r is greater than 15. In various embodiments, r is greater than 20. In various embodiments, r is greater than 25. In various embodiments, r is greater than 30. In various embodiments, r is greater than 35. In various embodiments, r is greater than 40. In various embodiments, r is greater than 45. In various embodiments, r is greater than 50. In various embodiments, r is between 10 and 50. In various embodiments, r is between 15 and 40. In various embodiments, r is between 20 and 30. In various embodiments, r is between 22 and 26.

Example Machine Learning Model

As described herein, generation of a small molecule compound involves the deployment of a machine learning model. In various embodiments, the machine learning model is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), or deep neural networks (DNN). In particular embodiments, the machine learning model is a neural network model. In particular embodiments, the machine learning model is a convolutional neural network (CNN), such as a CNN with the ResNet architecture.

Graph convolution networks achieve aggregation and sharing of information among nodes and edges by means of message passing. As the number of layers increases, information from many more hops away can be aggregated. Applying convolution neural network directly on the proposed adjacency tensor results in a similar information aggregation. As the depth of network increases, the field of view for each convolutional kernel increases, also allowing it to aggregate information of atoms from many hops away. Finally, this tensor graph embedding combines both node embedding and edge embedding in a single forward pass of the model.

The machine learning model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the machine learning model is trained using supervised learning algorithms or unsupervised learning algorithms.

In various embodiments, the machine learning model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the machine learning model are trained (e.g., adjusted) using the training data to improve the predictive power of the machine learning model.

In various embodiments, the input to the trained neural network is a graph adjacency tensor with a size of m×n×d. In various embodiments, values of m and n are determined according to the maximum number of atoms of the small molecule compound and d is the depth of feature channels. In various embodiments, the atom block, charge block, and the remaining valance channel include values in the diagonal section of the input cuboid, and may include zero values in the off-diagonal. In contrast, the bond block may have values in the off-diagonal. The output of the neural network is a vector of logits for all actions. The input graph tensor is passed through a residual tower including a single convolutional block followed by a number of residual blocks. The Conv block may contain the following operations:
A convolution operation by 128 filters of kernel size 3×3 with stride 1. A L2 weight decay with a regularization factor of 10-4 is also applied to the kernels;
A layer normalization operation;
A leaky rectifier nonlinear operation with a negative slope coefficient of 0.3.

In various embodiments, the residual block applies the following operation to the output of the Conv block:
An additional Conv block operation;
A skip connection;
A leaky rectifier nonlinear operation.

In various embodiments, the machine learning model includes a max pooling operation after every two consecutive residual blocks. A global max pooling followed by a dense layer can be added on top of the final residual block to generate logits. The size of the output logits may be the same as the size of action space.

Non-Transitory Computer Readable Medium

Also provided herein is a computer readable medium comprising computer executable instructions configured to implement any of the methods described herein. In various embodiments, the computer readable medium is a non-transitory computer readable medium. In some embodiments, the computer readable medium is a part of a computer system (e.g., a memory of a computer system). The computer readable medium can comprise computer executable instructions for implementing a machine learning model for the purposes of predicting a clinical phenotype.

Computing Device

The methods described above, including the methods of training and deploying machine learning models (e.g., classification model and/or regression model), are, in some embodiments, performed on a computing device. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

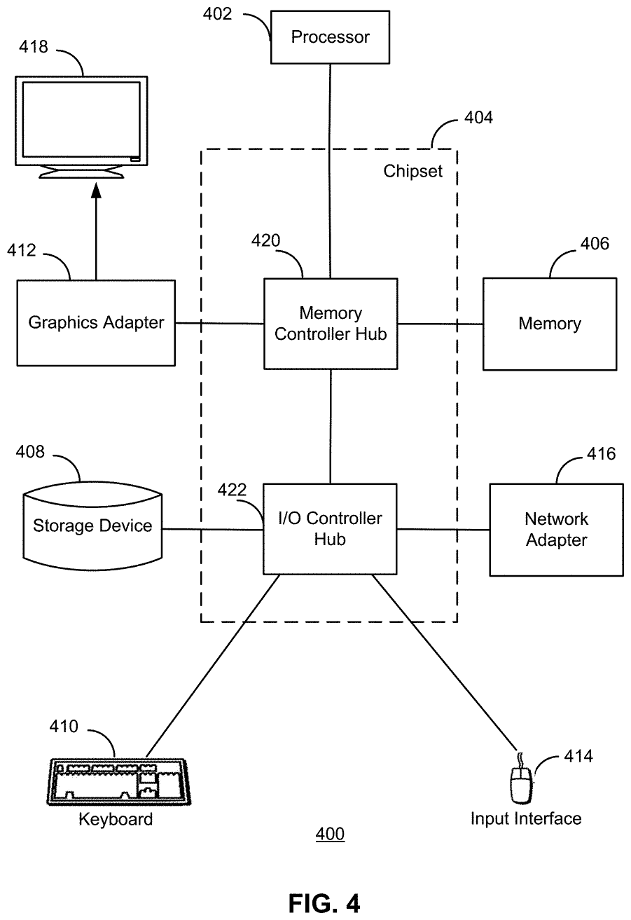

FIG. 4 illustrates an example computing device for implementing system and methods described in FIGS. 1A, 1B, 2A, 2B, 3A, and 3B. In some embodiments, the computing device 400 shown in FIG. 4 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, an input interface 414, and network adapter 416 are coupled to the I/O controller hub 422. Other embodiments of the computing device 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of input interface, a keyboard, or some combination thereof, and is used to input data into the computing device 400. In some embodiments, the computing device 400 may be configured to receive input (e.g., commands) from the input interface 414 via gestures from the user. The graphics adapter 412 displays images and other information on the display 418. The network adapter 416 couples the computing device 400 to one or more computer networks.

The computing device 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computing devices 400 can vary from the embodiments described herein. For example, the computing device 400 can lack some of the components described above, such as graphics adapters 412, input interface 414, and displays 418. In some embodiments, a computing device 400 can include a processor 402 for executing instructions stored on a memory 406.

The methods for generating new molecules can, in various embodiments, be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of a machine learning model of this invention. Such data can be used for a variety of purposes, such as patient monitoring, treatment considerations, and the like. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that is capable of recording and reproducing the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### EXAMPLES

### Example 1: Iterative Implementation of Neural Network Model Generates Valid Small Molecule Compounds

This example demonstrates the competitive generation capability of the proposed algorithm by comparing it to 11 state-of-the-art algorithms published recently. These compared algorithms cover the two main categories of generation methodology, including one-shot and sequential. The performance statistics of these algorithms are collected from experiments with the QM9, ZINC250k and ChEMBL datasets, thereby providing a comprehensive evaluation of their generation capability from short-length molecules to longer ones.

Dataset and Preprocessing

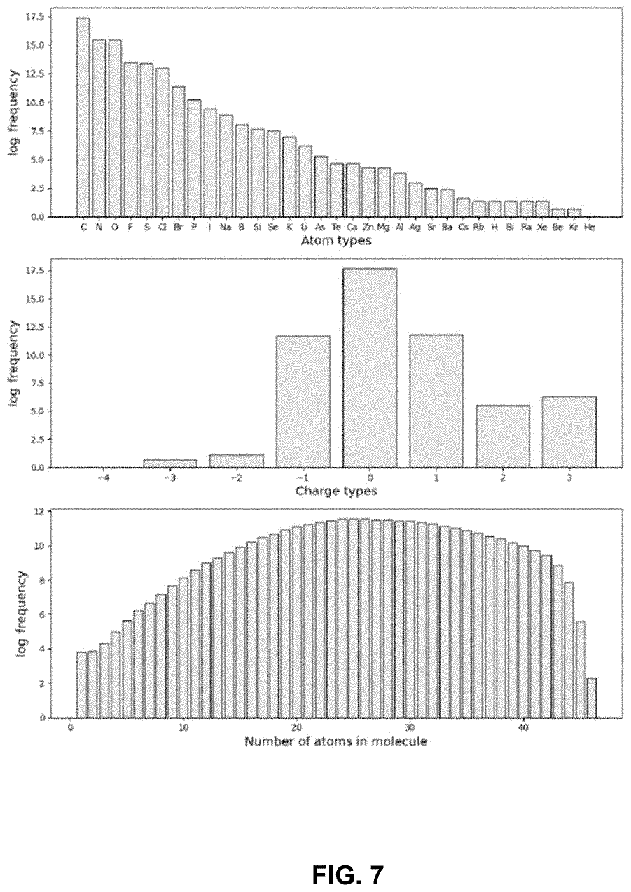

The QM9 dataset is provided in the work of Ramakrishnan, et al [30]. It contains 134 k organic molecules having up to 9 heavy atoms. ZINC250k dataset is made publicly available by Irwin, et al. [31]. This dataset comprises 250 k molecules with up to a maximum of 38 atoms. Similar to QM9, there are 9 atom types. For the ChEMBL dataset, 1.7 million molecules were downloaded from https://www.ebi.ac.uk/chembl/ with molecular weight up to 599. For all three datasets, the RDKit package was utilized to kekulize molecules and remove hydrogen atoms. Additionally, all molecules containing free radicals were removed. To determine the size of the graph adjacency tensor, a basic data analysis was conducted on the ChEMBL dataset to determine the maximum number of atoms, atom types, bond types and charge types. The analysis results are presented in FIG. 7 which shows the log frequency of atom types, charge types and the number of atoms per molecule in the downloaded ChEMBL dataset.

The 14 most frequently appearing atom types were selected including up to Potassium (K) atom which make up of more than 98% of molecules. Similarly, 5 charge types (−1 to 3) were selected, which cover more than 98% of charge types in the 1.7 million molecules. After kekulizing, there are only three bond types found in all molecules including single, double and triple. Additionally added to the bond types was a “No bond.” With these three numbers determined, the feature depth of the adjacency tensor was set to 24. Finally, the maximum number of atoms was chosen to be 41 which encapsulated more than 98% of molecules in the downloaded ChEMBL dataset. Therefore, the final size of the graph adjacency tensor was determined to be 41×41×24. This input size was fixed for all experiments.

Training molecules were stored in their SMILES string format. After kekulizing and removal of hydrogen atoms, every SMILES string was decomposed into a sequence of tensor-action pairs. These pairs were then concatenated to form the training dataset which was then randomly shuffled to break auto-correlation between samples. Subsequently, this dataset was split into training, validation and testing set with a ratio of 98%, 10% and 1% respectively. As a result, for the QM9 dataset, 512, 256 training samples and 45,312 val/testing samples were obtained. For ZINC250k, 11 million training samples and 480,0384 val/testing samples were obtained. The largest ChEMBL dataset yielded almost 100 million training samples and 1 million val/testing samples. Finally, the batch size for training was set to 128.

Evaluation Metrics

The generative capability of the proposed algorithm was evaluated by three metrics: the validity rate, uniqueness, and novelty. 10000 random molecules were generated to compute these metrics. The validity rate is the percentage of the valid molecules in the 10000 generated samples. A generated molecule is valid only if its corresponding adjacency tensor is valid. A generated adjacency tensor is chemically valid if it can be converted to a canonical SMILES string using RDKit. Let M be the list of canonical SMILES corresponding to the generated valid adjacency tensors, the uniqueness is then defined as the ratio set(M)/|M|. On the other hand, novelty measures the fraction of generated samples that are not present in the training dataset. Let N be the list of unique canonical smiles in the training dataset, novelty is computed as 1−|set(M)∩N|/|set(M)|.

Implementation Details

The proposed algorithm was implemented using Tensor-Flow [33] on a desktop platform having a single RTX 3080 GPU with 11 GB of memory. The neural network architecture for all three datasets was the same, consisting of a single Conv block followed by 10 residual blocks, a global max pooling layer, and a dense prediction layer. The neural network model has approximately 1.5 million trainable parameters. The loss function is the standard sparse cross-entropy loss, and Adam [34] optimizer was used for loss minimization. A staircase learning rate decay at every 200 k steps was adopted; the initial learning rate of 0.001 was reduced by a factor of 10, 50, and 100 at 200 k, 400 k, and 800 k steps respectively, and then remained the same until the end of training. The neural network was trained for 15 epochs, 4 epochs and 2 epochs on the QM9, ZINC, and ChEMBL dataset respectively. All training sessions were finished within 2 days.

Sequential generation starts with a random sampling of the number of atoms to be generated from a uniform distribution. The range of the uniform distribution is determined later based on the largest ChEMBL dataset. Upon determining this number, the procedure in FIG. 6 is followed. When sampling the bond adding action, in addition to the masking implemented in FIG. 6, additionally masked are the bond adding actions which violate the remaining valence of the connecting atoms. This leads to improved validity rate. Since each sub-graph corresponds to a substructure of a molecule, it is also possible to use chemical validity check tool such as RDKit to reject invalid bond adding to the substructure during sampling. This further pushes validity to 100%. Notably, this validity check at each generation step is not possible for SMILES string-based generation method.

### RESULTS AND DISCUSSION

The performance of the proposed algorithm on three benchmark datasets are summarized in Table 1. There are two validity results with one being verified by RDKit for every step of generation (re-sample until a valid action is generated) and the other relying completely on the trained model for generation. Generation with verification guarantees 100% validity rate. On the other hand, the validity rate without verification measures the capacity of a model to learn important structural and syntactic features among atoms and bonds that contribute to valid molecular graph generation. A sharp drop in this rate after removal of verification could indicate inadequate learning capacity, as a random action generation with infinite re-sampling could also guarantee 100% validity rate with verification.

TABLE 1
Result Summary

| | | |
|---|---|---|
| Valid | Unique | Novel |

| | verify | w/o verify | — | — |
|---|---|---|---|---|
| QM9 | 100.0% | 99.6% | 51.3% | 85% |
| ZINC | 100.0% | 90.5% | 90.2% | 100% |
| ChEMBL | 100.0% | 92.2% | 93.7% | 99.3% |

There is only a 0.4% drop in validity rate without verification for the QM9 dataset. This is expected as the maximum number of atoms is relatively small as compared to the other two datasets. There is less amount of “historical” information that the model needs to attend to for generating valid actions. As the number of atoms increases, the variation and the number of possible connections increase drastically, leading to a significant growth in complexity of the problem space. As a result, the validity rate drops by almost 10% on the ZINC dataset. However, with the number of training data samples increasing significantly from ZINC to ChEMBL, the validity rate increases again. Similarly, the uniqueness rate and novelty rate also increase with the number of training samples. This observation shows that larger datasets can better exploit the learning capacity of deep generative models. It is also worth mentioning that generating longer molecules would naturally lead to increase in the uniqueness and novelty, but often at the expense of reduced validity rate if a model has inadequate learning capacity.

The proposed algorithm was first compared to 8 state-of-the-art baselines on the QM9 dataset. All results of the compared methods were taken from their original publications. For conciseness of result presentation, the proposed algorithm implementing the neural network model disclosed herein is abbreviated as SEEM (structural encoding for embedding molecular structure). The result comparison for QM9 is summarized in Table 2.

TABLE 2
QM9 generation results

| Method | Valid | Unique | Novelty |
|---|---|---|---|
| CVAE [35] | 10.3% | 67.5% | 90.0% |
| GVAE [36] | 60.2% | 9.3% | 80.9% |
| GraphVAE [10] | 55.7% | 76.0% | 61.6% |
| RVAE [11] | 96.6% | NA | 97.5% |
| MolGAN [32] | 98.1% | 10.4% | 94.2% |
| GraphNVP [12] | 83.1% | 99.2% | 58.2% |
| GRF [13] | 84.8% | 66.0% | 58.6% |
| MGM [37] | 88.6% | 97.8% | 51.8% |
| SEEM | 99.6% | 51.3% | 85.0% |

The proposed SEEM has the highest validity rate among all compared methods. Interestingly, one-shot methods including RVAE and GraphNVP achieve the best novelty and uniqueness respectively. This could be attributed to the fact that generating all nodes and edges at once in an “unconditional” manner allows more flexibility as each node and edge generation from a learned latent representation can vary independently. In contrast, the SEEM model can be considered as a conditional generative model where each generation is conditioned on the previously generated sub-graph; there is much less flexibility in generation especially when the generated molecular graph is small.

The performance of SEEM improves significantly on the larger ZINC dataset with longer molecules, as shown in Table 3. SEEM is able to maintain a high validity rate while generating molecules with 100% novelty. In contrast, the one-shot based methods (GraphVAE, RVAE, Graph-NVP and GRF) suffer from model collapse as they do not have enough learning and generalization capability to generate longer molecules.

TABLE 3
ZINC250k generation results

| Method | Valid | Unique | Novelty |
|---|---|---|---|
| CVAE [14] | 0.0% | NA | 0.0% |
| GVAE [36] | 34.9% | NA | 2.9% |
| GraphVAE [10] | 13.5% | NA | NA |
| RVAE [11] | 34.9% | NA | 100% |
| GraphNVP [12] | 42.6% | 94.8% | 100% |
| GRF [13] | 73.4% | 53.7% | 100% |
| DGGen [14] | 89.2% | NA | 100% |
| GraphAF [17] | 68.0% | 99.1% | 100% |
| SEEM | 90.5% | 90.2% | 100% |

The final set of comparisons were made on the largest ChEMBL dataset where the performance of SEEM reaches its peak. This emphasizes that SEEM is belier able to handle larger training datasets (e.g., millions of training samples from the ChEMBL dataset) in comparison to state of the art methodologies. The results for the ChEMBL dataset is shown in Table 4. MGM [37] followed the pretraining setup of the BERT language model where random masking is applied to the output. Instead of masking tokens in a sentence, MGM randomly masks bonds and atoms in the output. Subsequently, a single GCN is trained to predict the masked action directly. The generation procedure corresponding to this formulation is fairly complex involving multiple steps of Gibbs sampling, whereas sampling for SEEM is much simpler. In this case, simplicity does lead to better result.

TABLE 4
ChEMBL generation results.

| Method | Valid | Unique | Novelty |
|---|---|---|---|
| MGM [37] | 84.9% | 100% | 72.2% |
| EGraphVAE [38] | 83.0% | 94.4% | 100% |
| DGGen [14] | 97.5% | NA | 90.0% |
| SEEM | 92.2% | 93.7% | 99.3% |

Altogether, disclosed herein is a neural network-based sequential graph generation algorithm. The presented algorithm is simple, easy to train and demonstrates excellent scalability in generating long molecules. Its competitive performance has been demonstrated by comparison to a number of recently published state-of-the-art baselines on three benchmark datasets. While the proposed algorithm maintains high validity rate across all datasets, its ability to generate unique and novel molecules improves significantly with larger datasets.

### REFERENCES

1. Anna Gaulton, Anne Hersey, Michal Nowotka, A Patricia Bento, Jon Chambers, David Mendez, Prudence Mutowo, Francis Atkinson, Louisa J Bellis, Elena Cibriin-Uhalte, et al. The chembl database in 2017. Nucleic acids research, 45(D1):D945-D954, 2017.
2. Jessica Vamathevan, Dominic Clark, Paul Czodrowski, Ian Dunham, Edgardo Ferran, George Lee, Bin Li, Anant Madabhushi, Parantu Shah, Michaela Spitzer, et al. Applications of machine learning in drug discovery and development. Nature Reviews Drug Discovery, 18(6):463-477, 2019.
3. Hongming Chen, Ola Engkvist, Yinhai Wang, Marcus Olivecrona, and Thomas Blaschke. The rise of deep learning in drug discovery. Drug discovery today, 23(6):1241-1250, 2018.
4. Pavel G Polishchuk, Timur I Madzhidov, and Alexandre Vamek. Estimation of the size of druglike chemical space based on gdb-17 data. Journal of computer-aided molecular design, 27(8):675-679, 2013.
5. DavidWeininger. Smiles, a chemical language and information system. 1. introduction to methodology and encoding rules. Journal of chemical information and computer sciences, 28(1):31-36, 1988.
6. Esben Jannik Bjerrum and Richard Threlfall. Molecular generation with recurrent neural networks (inns). arXiv preprint arXiv:1705.04612, 2017.
7. Esben Jannik Bjerrum. Smiles enumeration as data augmentation for neural network modeling of molecules. arXiv preprint arXiv:1703.07076, 2017.
8. Peter Ertl, Richard Lewis, Eric Martin, and Valery Polyakov. In silico generation of novel, drug-like chemical matter using the lstm neural network. arXiv preprint arXiv:1712.07449, 2017.
9. Francesca Grisoni, Michael Moret, Robin Lingwood, and Gisbert Schneider. Bidirectional molecule generation with recurrent neural networks. Journal of chemical information and modeling, 60(3):1175-1183, 2020.
10. Martin Simonovsky and Nikos Komodakis. Graphvae: Towards generation of small graphs using variational autoencoders. In International conference on artificial neural networks, pages 412-422. Springer, 2018.
11. Tengfei Ma, Jie Chen, and Cao Xiao. Constrained generation of semantically valid graphs via regularizing variational autoencoders. arXiv preprint arXiv:1809.02630, 2018.
12. Kaushalya Madhawa, Katushiko Ishiguro, Kosuke Nakago, and Motoki Abe. Graphnvp: An invertible flow model for generating molecular graphs. arXiv preprint arXiv:1905.11600, 2019.
13. Shion Honda, Hirotaka Akita, Katsuhiko Ishiguro, Toshiki Nakanishi, and Kenta Oono. Graph residual flow for molecular graph generation. arXiv preprint arXiv:1909.13521, 2019.
14. Yujia Li, Oriol Vinyals, Chris Dyer, Razvan Pascanu, and Peter Battaglia. Learning deep generative models of graphs. arXiv preprint arXiv:1803.03324, 2018.
15. Jiaxuan You, Bowen Liu, Rex Ying, Vijay Pande, and Jure Leskovec. Graph convolutional policy network for goal-directed molecular graph generation. arXiv preprint arXiv:1806.02473, 2018.
16. Dinghan Shen, Ash Celikyilmaz, Yizhe Zhang, Liqun Chen, Xin Wang, and Lawrence Carin. Hierarchically-structured variational autoencoders for long text generation. 2018.
17. Chence Shi, Minkai Xu, Zhaocheng Zhu, Weinan Zhang, Ming Zhang, and Jian Tang. Graphaf: a flow-based autoregressive model for molecular graph generation. arXiv preprint arXiv:2001.09382, 2020.
18. Thomas N Kipf and Max Welling. Semisupervised classification with graph convolutional networks. arXiv preprint arXiv:1609.02907, 2016.
19. Alec Radford, Karthik Narasimhan, Tim Salimans, and Ilya Sutskever. Improving language understanding by generative pre-training. 2018.
20. Jacob Devlin, Ming-Wei Chang, Kenton Lee, and Kristina Toutanova. Bert: Pre-training of deep bidirectional transformers for language understanding. arXiv preprint arXiv:1810.04805, 2018.
21. David Silver, Aja Huang, Chris J Maddison, Arthur Guez, Laurent Sifre, George Van Den Driessche, Julian Schrittwieser, Ioannis Antonoglou, Veda Panneershelvam, Marc Lanctot, et al. Mastering the game of go with deep neural networks and tree search. nature, 529(7587):484-489, 2016.
22. Kaiming He, Xiangyu Zhang, Shaoqing Ren, and Jian Sun. Deep residual learning for image recognition. In Proceedings of the IEEE conference on computer vision and pattern recognition, pages 770-778, 2016.
23. Paul Muller. Glossary of terms used in physical organic chemistry (iupac recommendations 1994). Pure and Applied Chemistry, 66(5):1077-1184, 1994.
24. Greg Landrum. Rdkit documentation. Release, 1(1-79):4, 2013.
25. Justin Gilmer, Samuel S Schoenholz, Patrick F Riley, Oriol Vinyals, and George E Dahl. Neural message passing for quantum chemistry. In International conference on machine learning, pages 1263-1272. PMLR, 2017.
26. Ashish Vaswani, Noam Shazeer, Niki Parmar, Jakob Uszkoreit, Llion Jones, Aidan N Gomez, Lukasz Kaiser, and Illia Polosukhin. Attention is all you need. In Advances in neural information processing systems, pages 5998-6008, 2017.
27. Volodymyr Mnih, Koray Kavukcuoglu, David Silver, Alex Graves, Ioannis Antonoglou, Daan Wierstra, and Martin Riedmiller. Playing Atari with deep reinforcement learning. arXiv preprint arXiv:1312.5602, 2013.
28. Jimmy Lei Ba, Jamie Ryan Kiros, and Geoffrey E Hinton. Layer normalization. arXiv preprint arXiv:1607.06450, 2016.
29. Bing Xu, Naiyan Wang, Tianqi Chen, and Mu Li. Empirical evaluation of rectified activations in convolutional network. arXiv preprint arXiv:1505.00853, 2015.
30. Raghunathan Ramakrishnan, Pavlo O Dral, Matthias Rupp, and O Anatole Von Lilienfeld. Quantum chemistry structures and properties of 134 kilo molecules. Scientific data, 1(1):1-7, 2014.
31. John J Irwin, Teague Sterling, Michael M Mysinger, Erin S Bolstad, and Ryan G Coleman. Zinc: a free tool to discover chemistry for biology. Journal of chemical information and modeling, 52(7):1757-1768, 2012.
32. Nicola De Cao and Thomas Kipf Molgan: An implicit generative model for small molecular graphs. arXiv preprint arXiv:1805.11973, 2018.
33. Martin Abadi, Paul Barham, Jianmin Chen, Zhifeng Chen, Andy Davis, Jeffrey Dean, Matthieu Devin, Sanjay Ghemawat, Geoffrey Irving, Michael Isard, et al. Tensorflow: A system for large-scale machine learning. In 12th {USENIX} symposium on operating systems design and implementation ({OSDI} 16), pages 265-283, 2016.
34. Diederik P Kingma and Jimmy Ba. Adam: A method for stochastic optimization. arXiv preprint arXiv:1412.6980, 2014.
35. Rafael G6mez-Bombarelli, Jennifer N Wei, David Duvenaud, Jose Miguel Hernindez-Lobato, Benjamin Sinchez-Lengeling, Dennis Sheberla, Jorge Aguilera-Iparraguirre, Timothy D Hirzel, Ryan P Adams, and Alin Aspuru-Guzik. Automatic chemical design using a data-driven continuous representation of molecules. ACS central science, 4(2):268-276, 2018.
36. Matt J Kusner, Brooks Paige, and Jose Miguel Hernindez-Lobato. Grammar variational autoencoder. In International Conference on Machine Learning, pages 1945-1954. PMLR, 2017.
37. Omar Mahmood, Elman Mansimov, Richard Bonneau, and Kyunghyun Cho. Masked graph modeling for molecule generation. Nature communications, 12(1):1-12, 2021.
38. Youngchun Kwon, Jiho Yoo, Youn-Suk Choi, Won-Joon Son, Dongseon Lee, and Seokho Kang. Efficient learning of non-autoregressive graph variational autoencoders for molecular graph generation. Journal of Cheminformatics, 11(1):1-10, 2019.

1. A method for generating one or more small molecule compounds, the method comprising:
obtaining a graph adjacency tensor, wherein the adjacency tensor comprises a representation of a small molecule compound;
iteratively applying a trained machine learning model to generate the small molecule compound, wherein each iteration comprises:
analyzing, using the machine learning model, the graph adjacency tensor to generate probabilities corresponding to available actions, wherein each probability indicates a likelihood of generating a valid substructure of a small molecule compound if a corresponding available action were taken;
selecting one of the available actions based on the probabilities;
updating the graph adjacency tensor with a value indicative of the selected action to generate an updated graph adjacency tensor,
wherein the trained machine learning model is trained using training examples indicating actions at individual steps of the small molecule building process.

2. A method for generating one or more small molecule compounds, the method comprising:
obtaining a graph adjacency tensor, wherein the adjacency tensor comprises a representation of a small molecule compound;
iteratively applying a trained machine learning model to generate the small molecule compound, wherein each iteration comprises:
analyzing, using the machine learning model, a subgraph of the graph adjacency tensor to generate probabilities corresponding to available actions, the subgraph representing a substructure of the small molecule compound, wherein each probability indicates a likelihood of generating a valid substructure of a small molecule compound if a corresponding available action were taken, wherein the available actions comprise adding an atom, adding a bond, or assigning a charge to an atom;
selecting one of the available actions based on the probabilities;
updating the graph adjacency tensor with a value indicative of the selected action to generate an updated graph adjacency tensor,
wherein the trained machine learning model is trained using training examples indicating actions at individual steps of the small molecule building process, wherein the training examples are generated by:
obtaining a plurality of training small molecule compounds;
for each of one or more training small molecule compounds of the plurality, generating a plurality of subgraph-action pairs for the training small molecule compound by:
decomposing the training small molecule compound into a sequence of actions for generating the training small molecule compound;
generating graph adjacency tensor subgraphs for actions in the sequence of actions; and
pairing each action with a corresponding graph adjacency tensor subgraph;
randomly shuffling subgraph-action pairs to break autocorrelations; and
assigning randomly shuffled subgraph-action pairs to the training examples.

3. The method of claim 1, wherein the available actions comprise adding an atom, adding a bond, or assigning a charge to an atom.

4. The method of claim 1, wherein the iterative application of the trained machine learning model terminates after the small molecule compound comprises at least a threshold number of atoms.

5. The method of claim 4, wherein the threshold number of atoms is at least 5, at least 10, at least 20, at least 30, at least 40, or at least 50 atoms.

6. The method of claim 1, wherein selecting one of the available actions based on the probabilities comprises selecting an available action corresponding to a highest probability.

7. The method of claim 1, wherein the selected action comprises adding an atom, and wherein updating the graph adjacency tensor with a value indicative of the selected action comprises updating a diagonal of the graph adjacency tensor with a value indicative of the added atom.

8. The method of claim 1, wherein the selected action comprises adding a bond, and wherein updating the graph adjacency tensor with a value indicative of the selected action comprises updating an upper portion of the graph adjacency tensor with a value indicative of the added bond.

9. The method of claim 1, wherein the selected action comprises assigning a charge to an atom, and wherein updating the graph adjacency tensor with a value indicative of the selected action comprises updating a diagonal of the graph adjacency tensor with a value indicative of the assigned charge.

10. The method of claim 1, wherein the graph adjacency tensor comprises m×n×d dimensions.

11. The method of claim 10, wherein m or n represents a pre-determined maximum number of atoms of the small molecule compound.

12. The method of claim 11, wherein each of m or n is between 20 and 60, and d is between 20 and 100.

13. (canceled)

14. (canceled)

15. The method of claim 1, wherein analyzing, using the trained machine learning model, the graph adjacency tensor to generate probabilities corresponding to available actions further comprises:
determining a subgraph of the graph adjacency tensor, the subgraph representing a substructure of the small molecule compound; and
analyzing the subgraph using the trained machine learning model to predict probabilities corresponding to available actions.

16. The method of claim 15, wherein the subgraph has dimensions of p×q×r, and each of the dimensions p×q×r of the subgraph are smaller than or equal to each of corresponding dimensions m×n×d of the graph adjacency tensor.

17. The method of claim 16, wherein each of p, q or r is between 1 and 5.

18. (canceled)

19. (canceled)

20. The method of claim 1, wherein the training examples used to train the machine learning model are generated by:
obtaining a plurality of small molecule compounds;
for each of one or more small molecule compounds of the plurality, generating a plurality of subgraph-action pairs for the small molecule compound by:
decomposing the small molecule compound into a sequence of actions for generating the small molecule compound;
generating graph adjacency tensor subgraphs for actions in the sequence of actions; and
pairing each action with a corresponding graph adjacency tensor subgraph;
randomly shuffling subgraph-action pairs to break autocorrelations; and
assigning randomly shuffled subgraph-action pairs to training examples.

21-25. (canceled)

26. The method of claim 1, wherein at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% of the generated small molecule compounds are chemically valid.

27. (canceled)

28. (canceled)

29. The method of n m claim 1, wherein at least 85%, at least 90%, at least 91%, at least 92%, at least 93%, at least 94%, at least 95%, at least 96%, at least 97%, at least 98%, or at least 99% of the generated small molecule compounds are novel.

30. (canceled)

31. (canceled)

32. The method of claim 1, wherein the trained machine learning model is a trained neural network.

33. A non-transitory computer readable medium comprising instructions that, when executed by a processor, cause the processor to:
obtain a graph adjacency tensor, wherein the adjacency tensor comprises a representation of a small molecule compound;
iteratively apply a trained machine learning model to generate the small molecule compound, wherein each iteration comprises:
analyzing, using the trained machine learning model, the graph adjacency tensor to generate probabilities corresponding to available actions, wherein each probability indicates a likelihood of generating a valid substructure of a small molecule compound if a corresponding available action were taken;
selecting one of the available actions based on the probabilities;
updating the graph adjacency tensor with a value indicative of the selected action to generate an updated graph adjacency tensor,
wherein the trained machine learning model is trained using training examples indicating actions at individual steps of the small molecule building process.

34-62. (canceled)
